(19) United States
(12) Patent Application Publication
(10) Pub. No.: US 2023/0310553 A1
(43) Pub. Date: Oct. 5, 2023

(54) WOUND HEALING AND TISSUE REPAIR COMPOSITION AND METHOD USING LOW MOLECULAR WEIGHT COLLAGEN AND BIOACTIVE GLASS

(71) Applicant: GEORGE D. PETITO, BETHLEHEM, PA (US)

(72) Inventor: GEORGE D. PETITO, BETHLEHEM, PA (US)

(21) Appl. No.: 18/128,866

(22) Filed: Mar. 30, 2023

Related U.S. Application Data
(60) Provisional application No. 63/325,260, filed on Mar. 30, 2022.

(51) Int. Cl. A61K 38/39; A61K 33/42; A61K 33/06

(52) U.S. Cl. CPC A61K 38/39; A61K 33/42; A61K 33/06

(57) ABSTRACT

A wound healing and tissue repair composition with low molecular weight collagen and bioactive glass is provided. The wound healing and tissue repair composition with low molecular weight collagen and bioactive glass is a medicinal composition for facilitating the growth, protection and healing of tissues and cells in animals and humans. The main ingredients of the formulated composition are collagen having a molecular weight of about 1,000 Da or less and bioactive glass, which can be combined with polysulfated glycosaminoglycans, hyaluronic acid or salts thereof, or a glucosamine salt, and mixtures thereof. The composition may include collagen from multiple sources, such as bovine sourced collagen or marine sourced collagen and may further include hydrolyzed whey. The collagen may be hydrolyzed collagen, native collagen, or a mixture thereof.

### CROSS-REFERENCE TO RELATED APPLICATION

This application claims the benefit of U.S. Provisional Patent Application No. 63/325,260, filed on Mar. 30, 2022.

### BACKGROUND

### 1. Field

A wound healing and tissue repair composition and method using low molecular weight collagen and bioactive glass relates to medical compositions, and particularly, to a method and composition using hydrolyzed collagen having a molecular weight of about 1,000 Daltons (Da) or less and bioactive glass.

### 2. Description of the Related Art

Just as nature has provided the skin as a barrier for protection, it has also provided mechanisms for skin repair. Depending upon the nature of the injury, this repair process may take hours, days, months, or even years. Many factors determine the length of times it takes for injured skin to heal. Pathogenic contaminants may enter the body through the wound until the skin's integrity is restored. For this reason, it is desirable to heal open wounds as quickly as possible.

Open wounds in the skin are a potential gateway for infectious or contaminating material to enter the body. The skin is a protective barrier to external contaminants. When the skin is damaged with an open breach, these contaminants are free to enter the body. Once inside the body, these contaminants may have effects of varying degrees, but almost always become more difficult to treat, and consequently slow the healing process of the original wound.

To fight infection, wound management traditionally involves an initial cleansing of the affected area to remove any contaminants such as dirt, clothing particles, or other debris. Damaged tissues and foreign materials are removed when necessary, and antiseptic agents are applied to sterilize the injured area. Sterile dressings are often applied, and periodically changed, to keep the injured area as clean and sterile as possible. Complex biological mechanisms occur during the healing process such as chemical signals attracting fibroblast cells to the wound site which ultimately generate connective structures mainly of collagen. Endothelial cells generate new blood capillaries that nurture the new growth. The cell growth continues until the open wound is filled by forming permanent new tissue.

Because shortened periods of healing result in shortened exposure time, it would be beneficial to have any open wound heal as quickly as possible.

Traditional methods of wound healing have disadvantages, such as incomplete pigment removal, non-selective tissue destruction, and unsatisfactory cosmetic results, such as atrophic or hypertrophic scarring.

Thus, a wound healing and tissue repair composition and method solving the aforementioned problems is desired.

### SUMMARY

The wound healing and tissue repair composition and method using low molecular weight collagen facilitates healing of damaged tissues, promotes tissue and cell growth, protects cells and tissues, and reduces scar tissue. The composition includes proteinaceous amino acids, such as native collagen and/or hydrolyzed collagen, and bioactive glass. In some embodiments, hydrolyzed collagen may be combined with native collagen, further sources of amino acids, and/or at least one other therapeutic agent. For example, further sources of amino acids may include gelatins, whey, or hydrolyzed whey and the therapeutic agent may be a polysulfated glycosaminoglycan, a glucosamine salt, or mixtures thereof.

Accordingly, in one embodiment, the present subject matter relates to a composition for wound healing and tissue repair comprising low molecular weight collagen and bioactive glass, wherein the low molecular weight collagen has a molecular weight of about 10-1,000 Daltons.

In a further embodiment, the present compositions comprise hydrolyzed collagen, which can not only serve as the main therapeutic component, but can act as a pharmaceutical carrier when combined with a medicinal agent. By way of non-limiting example, such a medicinal agent can be selected from the group consisting of hyaluronic acid and salts thereof, a polysulfated glycosaminoglycan, a glucosamine salt, and mixtures thereof to aid tissue and cells to grow and wounds to heal as quickly as possible, or salicylic acid for treating warts. When combined with such a medicinal agent, the collagen in the present compositions can potentially enhance skin penetration of the medicinal agent, when topically applied.

In another embodiment, the present subject matter relates to a composition for wound healing and tissue repair comprising bioactive glass and hydrolyzed collagen, the hydrolyzed collagen comprising bovine sourced hydrolyzed collagen, marine sourced hydrolyzed collagen, and hydrolyzed whey, wherein one or more of the bovine sourced hydrolyzed collagen and the marine sourced hydrolyzed collagen comprise low molecular weight hydrolyzed collagen having a molecular weight of about 100-1,000 Daltons.

In an embodiment, the native collagen and/or hydrolyzed collagen in the composition is entirely or partially a low molecular weight collagen. The low molecular weight collagen may comprise one or more sources of collagen having a molecular weight of about 1,000 Da or less, about 500 Da or less, or about 400 Da or less. In an embodiment, the low molecular weight collagen may comprise collagen having a molecular weight ranging from about 10 Da to about 1,000 Da. In an embodiment, the low molecular weight collagen may comprise collagen having a molecular weight of between about 1,000 Da and about 100 Da, between about 500 Da and about 100 Da, or between about 400 Da and about 100 Da. In a further embodiment, the low molecular weight collagen may consist of one or more sources of collagen having a molecular weight of about 1,000 Da or less, about 500 Da or less, or about 400 Da or less. In a further embodiment, the low molecular weight collagen may consist of one or more sources of collagen having a molecular weight of between about 1,000 Da and about 100 Da, between about 500 Da and about 100 Da, or between about 400 Da and about 100 Da.

These and other features of the present subject matter will become readily apparent upon further review of the following specification.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

As used herein, “hydrolyzed collagen” is defined as a collagen hydrolysate polypeptide having a molecular weight lower than native collagen, i.e., in the 10 to 300,000 Daltons range, and is derived by hydrolysis.

As used herein, “bioactive glass” refers to a calcium silicate composed of at least SiO₂ and CaO. In certain embodiments, the bioactive glass may also include sodium (Na₂O), phosphorous (P₂O₅), potassium (K₂O), Magnesium (MgO), or a combination thereof.

As used herein, “hyaluronic acid” (HA) is rapidly hydrolyzed upon contact with treated tissue surfaces to monosaccharides, glucuronic acid and N-acetyl glucosamine. Chemical binding is enhanced with the use of hydrolyzed collagen, i.e., it is chemotactic. Hyaluronic acid can be used via injection into a joint for its anti-inflammatory effect to relieve pain and suffering. This curative effect is inherently terminated when hyaluronic acid is consumed by the healing body.

As used herein, “glycosaminoglycans” (GAGs) are polysaccharides found in vertebrate and invertebrate animals. Several GAGs have been found in tissues and fluids of vertebrate animals. The known GAGs are chondroitin sulfate, keratin sulfate, dermatan sulfate, hyaluronic acid, heparin, and heparin sulfate. GAGs and collagen are the major structural elements of all animal tissue. Their synthesis is essential for proper repair, treatment, protection, and maintenance of all tissues.

As used herein, “chondroitin sulfate”, a polysulfated GAG, is a linear polymer occurring in several isomers, named for the location of the sulfate group. Chondroitin-4 sulfate is found in nasal and tracheal cartilages of bovines and porcines. It is also found in the bones, flesh, blood, skin, umbilical cord, and urine of these animals. Chondroitin-6 sulfate has been isolated from the skin, umbilical cord, and cardiac valves of the aforementioned animals. Chondroitin-6 sulfate has the same composition, but slightly different physical properties from the chondroitin-4 sulfate. These are the most common isomers used herein. The polymers are also known as polysulfated glycosaminoglycans (PSGAGs), chondroitin polysulfate sodium, chondrin, sodium chondroitin polysulfate, and sodium chondroitin sulfate. For consistency, the term, “chondroitin sulfate”, will be recited for all chondroitin sulfate isomers throughout this specification. Chondroitin sulfate is involved in the binding of collagen and is also directly involved in the retention of moisture in the tissue. These are both valuable chemical properties that aid the healing process.

As used herein, “subject” may refer to any animal, including but not limited to human beings and other mammals.

### Wound Healing and Tissue Repair Composition

The wound healing and tissue repair composition includes proteinaceous amino acids, such as native collagen and/or hydrolyzed collagen, which may facilitate tissue and cell growth, as well as wound healing and tissue repair. Hydrolyzed collagen is a collagen hydrolysate polypeptide having a molecular weight lower than native collagen. Hydrolyzed collagen may be obtained by hydrolysis of native collagen. This may be accomplished by one of four methods: (1) alkaline hydrolysis; (2) enzymatic hydrolysis; (3) acid hydrolysis; and (4) synthetically, by fermentation. Any of these methods can be used to derive the hydrolyzed collagen from a collagen source.

In addition to native collagen and/or hydrolyzed collagen, the proteinaceous amino acids can include whey and/or hydrolyzed whey. The native collagen and the hydrolyzed collagen can be derived from any suitable collagen source. The collagen source can be, for example, a bovine (skin and tendon preferred), a porcine, a reptile, a marine, an avian, or a synthetic source. The collagen can be derived from a combination of two or more collagen sources, e.g., a bovine source and a marine source. The marine source can include any fish. In an embodiment, the marine source includes salmon, tilapia, or a combination of salmon and tilapia. The types of amino acid constituents and their sequences determine the beneficial healing qualities of hydrolyzed collagen. Hydroxylysine and hydroxyproline are amino acids found only in collagen and in no other medical protein hydrolysates. Hydroxylysine is typically found in concentrations from 0.7 to 1.2 wt. % in hydrolyzed collagen.

The native collagen and/or hydrolyzed collagen can be derived from bovine, porcine, and/or marine sources, for example. Bovine and porcine hydrolyzed collagen have high glycine, proline, hydroxyproline, and glutamic acid content. They also display hydrophilic properties. Bovine hydrolyzed collagen, for example, demonstrates strong hydrophilic properties and when used to treat wound sites demonstrates increased perfusion and epithelialization and decreased inflammatory reaction. In contrast, marine derived, i.e., marine sourced, hydrolyzed collagen has a different amino acid profile, with higher levels of aspartic acid, cysteine, glutamine, citruline, and asparagine.

While hydrolyzed collagen of any molecular weight may be used, the hydrolyzed collagen can be Low Molecular Weight hydrolyzed collagen (hereinafter “LMW hydrolyzed collagen”) having a molecular weight of about 1,000 Da or less. The LMW hydrolyzed collagen can have increased bioavailability. In an embodiment, the LMW hydrolyzed collagen may comprise collagen having a molecular weight ranging from about 10 Da to about 1,000 Da. The LMW hydrolyzed collagen may comprise hydrolyzed collagen having a molecular weight of about 1,000 Da or less, about 500 Da or less, or about 400 Da or less. In a further embodiment, the LMW hydrolyzed collagen may comprise hydrolyzed collagen having a molecular weight of between about 1,000 Da and about 100 Da, between about 500 Da and about 100 Da, or between about 400 Da and about 100 Da. In a further embodiment, the LMW hydrolyzed collagen may consist of hydrolyzed collagen having a molecular weight of about 1,000 Da or less, about 500 Da or less, or about 400 Da or less. In a further embodiment, the LMW hydrolyzed collagen may consist of hydrolyzed collagen having a molecular weight of between about 1,000 Da and about 100 Da, between about 500 Da and about 100 Da, or between about 400 Da and about 100 Da.

The LMW hydrolyzed collagen can be prepared by partially hydrolyzing native collagen in any suitable manner known in the art. Preferably, raw materials from one or more collagen sources are ground to a powder, enzymatically treated, fractionated, and purified to obtain high molecular weight hydrolyzed collagen. Bulk fractionation methods known in the art can be used. The raw materials can include, for example, fat, blood, tissue, and/or bone marrow from one or more collagen sources. Raw material from fish can further include, e.g., fish heads and/or fins.

In an alternative to the use of LMW hydrolyzed collagen, LMW native collagen may be used. The LMW native collagen may be Type 3 collagen. The LMW native collagen may comprise collagen having a molecular weight of about 1,000 Da or less, about 500 Da or less, or about 400 Da or less. In a further embodiment, the LMW native collagen may comprise collagen having a molecular weight of between about 1,000 Da and about 100 Da, between about 500 Da and about 100 Da, or between about 400 Da and about 100 Da. In a further embodiment, the LMW native collagen may consist of collagen having a molecular weight of about 1,000 Da or less, about 500 Da or less, or about 400 Da or less. In a further embodiment, the LMW native collagen may consist of collagen having a molecular weight of between about 1,000 Da and about 100 Da, between about 500 Da and about 100 Da, or between about 400 Da and about 100 Da.

The use of Type 3 collagen may provide unique benefits, as Type 3 collagen may be found in short peptide fragments having a molecular weight of about 1,000 Da or less. Thus, Type 3 native collagen may be used as LMW collagen without the need for hydrolyzing. Type 3 collagen may also provide further unique benefits through cross-linking with other components of the compositions disclosed herein, including Type 1 collagen, hyaluronic acid, glucosamine, elastin, and the like.

The wound healing and tissue repair composition also includes bioactive glass. The bioactive glass may have an average particle size (diameter) between about 10 μm and about 90 μm. In another embodiment, the bioactive glass may have an average particle size between about 35 μm and about 45 μm. The bioactive glass may make up anywhere from about 0.1% to about 50% of the wound healing and tissue repair compositions.

In certain embodiments, the bioactive glass may make up between about 0.1% and about 2.0% of the wound healing and tissue repair compositions, including about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0% of the wound healing and tissue repair composition. In other embodiments, the bioactive glass may make up between about 0.1% and about 10.5% of the wound healing and tissue repair composition, including about 0.1%, about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, or about 10.5% of the wound healing and tissue repair composition. In still further embodiments, the bioactive glass may make up between about 1% and about 50% of the wound healing and tissue repair composition, including about 1%, about 5%, about 10%, about 15%, about 20%, about 25%, about 30%, about 35%, about 40%, about 45%, or about 50% of the wound healing and tissue repair composition.

The concentration of bioactive glass in the wound healing and tissue repair composition may be selected based upon the intended application of the composition. For example, when formulating a wound healing and tissue repair composition for use in healing soft tissues or hard tissues, the bioactive glass may make up between about 5% and about 15% of the wound healing and tissue repair composition. In a particular embodiment, when formulating a wound healing and tissue repair composition for use in healing soft tissues or hard tissues, the bioactive glass may make up about 10% of the wound healing and tissue repair composition. In a further example, when formulating a wound healing and tissue repair composition for topical dermatological applications, the bioactive glass may make up between about 0.1% and about 3% of the wound healing and tissue repair composition. In a particular embodiment, when formulating a wound healing and tissue repair composition for topical dermatological applications, the bioactive glass may make up about 2% or less of the wound healing and tissue repair composition.

In certain embodiments, the bioactive glass may comprise about 45 wt % SiO₂, about 24.5 wt % CaO, about 24.5 wt % Na₂O, and about 6.0 wt % P₂O₅. In certain embodiments, the ratio of CaO:P₂O₅ is selected to enhance surface activity in a physiological environment, which can potentially enhance bonding to soft tissue and stimulate fibroblasts. Further, this configuration of the bioactive glad can potentially provide a relatively high dissolution rate, thereby enhancing soft tissue repair.

In certain embodiments, the bioactive glass particle size may be about 5 μm, about 10 μm, about 15 μm, about 20 μm, about 25 μm, about 30 μm, about 35 μm, about 40 μm, about μm, about 50 μm, about 55 μm, about 60 μm, about 65 μm, about 70 μm, about 75 μm, about 80 μm, about 85 μm, about 90 μm, or about 95 μm.

In certain embodiments, the bioactive glass may have a density of about 2.70 g/cc.

The present composition may include a combination of hydrolyzed collagen and native collagen. Combining native collagen with hydrolyzed collagen may enhance the bacteriostatic effects, as well as the cellular repair, wound healing, and tissue repair properties, of the composition. The different molecular weights of the native collagen and the hydrolyzed collagen in the composition may facilitate better control over absorption amount and absorption time of the composition, as well as the degradation time of the composition. For example, by varying the ratio of the native collagen to hydrolyzed collagen, various absorption rates and degradation rates may be achieved. Preferably, the composition includes a combination of LMW hydrolyzed collagen and native collagen. The native collagen may be LMW native collagen. Soluble and/or insoluble native collagen may be used.

The composition may include about 1% by weight to about 99% by weight hydrolyzed collagen. For example, the composition may include about 10% by weight to about 85% by weight hydrolyzed collagen or about 20% by weight to about 75% by weight hydrolyzed collagen, or about 30% by weight to about 65% by weight hydrolyzed collagen. In an embodiment, the hydrolyzed collagen is LMW hydrolyzed collagen. The composition may include about 0.1% by weight to about 65% by weight of soluble or insoluble native collagen. For example, the composition may include about 2% by weight to about 45% by weight of soluble or insoluble native collagen, or about 10% by weight to about 30% by weight of soluble or insoluble native collagen. The composition may include hydrolyzed collagen cross-linked with native collagen. For example, the composition may include about 0.1% by weight to about 65% by weight insoluble or soluble native collagen crosslinked with LMW hydrolyzed collagen. Other amounts below and above these ranges may be used.

The composition may include about 1% by weight to about 99% by weight collagen. For example, the composition may include about 10% by weight to about 85% by weight collagen or about 20% by weight to about 75% by weight collagen, or about 30% by weight to about 65% by weight collagen. The collagen is preferably LMW collagen. The composition may include about 0.1% by weight to about 65% by weight of soluble or insoluble native collagen. For example, the composition may include about 2% by weight to about 45% by weight of soluble or insoluble native collagen, or about 10% by weight to about 30% by weight of soluble or insoluble native collagen. The composition may include hydrolyzed collagen cross-linked with native collagen. For example, the composition may include about 0.1% by weight to about 65% by weight insoluble or soluble native collagen crosslinked with LMW hydrolyzed collagen. Other amounts below and above these ranges may be used.

In an embodiment, the composition can include mixtures of collagen from different collagen sources. For example, the composition can include bovine sourced collagen, marine sourced collagen, and whey protein. Alternatively, the composition can include bovine sourced collagen and marine sourced collagen. According to one embodiment, the proteinaceous amino acids in the composition can include bovine sourced hydrolyzed collagen, marine sourced hydrolyzed collagen, and hydrolyzed whey protein. The composition can further include elastin. Hydrolyzed whey protein offers another alternative amino acid profile, rich in glutamic acid, isoleucine, leucine, threonine, tyrosine, and valine.

Varying the source of the amino acids in the composition can control the chemotactic, hydrophilic, and cell proliferative properties of the composition. These properties may be manipulated in order to optimize the wound healing and/or tissue repair process. This optimization may adjust the timing and balance of stimulating the inflammatory and vascular systems, as well as involvement of connective tissues and epithelial cells.

For example, a heavily exudative or wet wound can be treated, at least initially, with a highly hydrophilic composition, including at least about 50% by weight bovine sourced hydrolyzed collagen (e.g., about 50% to about 60% by weight bovine sourced hydrolyzed collagen), at least about 20% by weight marine sourced hydrolyzed collagen (e.g., about 20% to about 30% by weight marine sourced hydrolyzed collagen), and up to about 30% by weight hydrolyzed whey protein (e.g., about 5% to about 30% by weight hydrolyzed whey protein). Elastin may be added to this composition (e.g., up to about 20%) during the closing phase of wound treatment to improve tensile strength and reduce scar formation. In contrast, a dry wound can be treated with at least about 50% by weight marine sourced hydrolyzed collagen, about 20% by weight or less hydrolyzed whey, about 10% by weight or less bovine sourced hydrolyzed collagen, and about 5% by weight or less elastin.

In a further embodiment, the composition may comprise bovine sourced hydrolyzed collagen, marine sourced hydrolyzed collagen and hydrolyzed whey. The composition may include about 50% to about 70% bovine sourced hydrolyzed collagen, about 20% marine sourced hydrolyzed collagen, and about 10% bovine derived hydrolyzed whey. Some or all of the hydrolyzed collagen in this composition may be LMW hydrolyzed collagen.

One or more additional therapeutic agents may be included in the composition to further speed the healing process, decrease scarring and increase tissue strength. Examples of suitable therapeutic agents that may be combined with the hydrolyzed collagen are glycosaminoglycans (GAGs), particularly GAGs useful for cellular repair. Antimicrobials may also be included in the composition to further enhance its bacteriostatic quality, as can antibiotics (such as tetracycline, streptomycin, and cephalosporin) and antibacterials (such as iodine, parachlorometaxylenol, and chlorhexidine gluconate or acetate). The composition may further include lipoic acid, alpha lipoic acid, one or more vitamins (e.g., vitamin A, vitamin B12, vitamin C, vitamin D, vitamin E), omega compounds or omega-3 fatty acid compounds (e.g., α-linolenic acid “ALA”, Eicosapentaenoic acid “EPA”, Docosahexaenoic acid “DHA”), antioxidants (e.g., superoxide dismustase, glutathione peroxidase, glutathione reductase), chitosan, and/or phytochemicals (e.g., zeaxanthin, lutein). Also, it has been established that hydrolyzed collagen used as a carrier in powder form, paste or a lyophilized foam has hemostatic qualities when combined with thrombin to improve healing of wounds.

Hydrolyzed collagen in combination with GAGs, specifically a PSGAG (such as chondroitin sulfate), can be useful for the prevention and treatment of wound diseases. The hydrolyzed collagen combines with a PSGAG to bond or adhere selectively to tissue, resulting in interference with and/or displacement of bacterial or other infectious agents. In addition, the combination product may exhibit anti-enzyme activity or the ability to inhibit enzyme activity.

The hydrolyzed collagen accelerates the healing process by allowing an injured tissue to repair itself by producing and remodeling more collagen and other proteoglycans (PGs). The building blocks for collagen production are the amino acids found in hydrolyzed collagen. Hyaluronic acid and other proteoglycans (PGs) provide the framework for collagen production to follow. The PGs hold water to provide an excellent environment for healing of the tissue to begin. When in the wound site, any unused collagen that was produced is simply degraded to the amino acid. The rate-limiting step in the production of collagen is the conversion of glucose to glucosamine for the production of hyaluronic acid and other glycosaminoglycans (GAGs).

The composition can include one or more therapeutic agents, such as an antibiotic, and/or one or more additives, such as glutamine, glycosaminoglycans, zinc, alginates, cellulose, and/or honey.

These are simplified examples, as wound healing and tissue repair are complex and wound specific. More complicated wounds, such as diabetic wounds, are treated using customized treatment regimens. For example, a diabetic wound can initially be treated as a wet wound, but with significantly more emphasis on hydrolyzed whey in the early treatment composition. During the later closing phase of wound treatment, the composition can be shifted to up to about 40% by weight of marine sourced hydrolyzed collagen, up to about 25% by weight of bovine sourced hydrolyzed collagen, and up to about 5% by weight of elastin.

Further examples of this wound healing and tissue repair composition optimized for different applications include: about 70% bovine sourced hydrolyzed collagen and about 30% marine sourced hydrolyzed collagen; about 50% bovine sourced hydrolyzed collagen, about 30% marine sourced hydrolyzed collagen, and about 20% hydrolyzed whey; about 40% bovine sourced hydrolyzed collagen, about 20% marine sourced hydrolyzed collagen, about 20% hydrolyzed whey, and about 20% elastin; about 20% bovine sourced hydrolyzed collagen, about 40% marine sourced hydrolyzed collagen, about 20% hydrolyzed whey, and about 20% elastin; about 20% bovine sourced hydrolyzed collagen, about 40% marine sourced hydrolyzed collagen, and about 40% hydrolyzed whey; and about 30% bovine sourced hydrolyzed collagen, about 30% marine sourced hydrolyzed collagen, about 30% hydrolyzed whey, and about 10% elastin. The bovine hydrolyzed collagen can have a molecular weight of about 500 Daltons to about 10,000 Daltons. The porcine hydrolyzed collagen can have a molecular weight of about 1,000 Daltons to about 15,000 Daltons. The salmon hydrolyzed collagen can have a molecular weight of about 100 Daltons to about 10,000 Daltons. The elastin can have a molecular weight of about 35,000 Daltons to about 145,000 Daltons.

The composition may be used to heal topical and/or internal wound sites. For example, the composition may be used prior to and after surgery to minimize cell damage and to expedite wound healing and/or tissue repair. The composition may be useful during surgery to foster separation of tissue to prevent adhesion formation. The composition may be used as a filler for a wound site and remain in the wound site as it heals, becoming part of the granulated tissue.

The composition may be useful for applications relating to cosmetic usage generally and plastic surgery, e.g., as a filler for lines and wrinkles formed in the skin.

The composition may take a physical form used in topical administration, such as a gel, spray, powder, paste, foam, film for incorporation in a dressing bandage, or a topically applied patch. The composition may take a physical form used in internal administration, such as an injectable liquid or an orally ingestible liquid.

The composition may be formulated for use as a food or a medical food. Medical foods are foods that are formulated to be consumed or administered under supervision of a physician and which are intended for the specific dietary management of a disease condition for which distinctive nutritional requirements are established by medical evaluation. The composition formulated for use as a medical food may be formulated for oral consumption or for tube feeding.

The powder composition will preferably have a moisture content of about 2-10 wt. % and a pH range of about 5.5 to about 6.5. The powder composition will have an ash content of about 2.5 wt. % or less and an isotonic point of about 5.0 to about 6.5. In use, the powder composition may be the preferred physical form for use with irregularly shaped wounds. Tunnel wounds, flaps, and other non-conformative sites may be managed with the powder composition because it easily conforms to any shape wound and may be applied by a poofer bottle or otherwise blown into difficult to reach wound sites. The powder is especially useful in wounds having a large amount of exudate, as the powder can absorb nearly 30 times its own weight. As the powder absorbs the exudate, a gel is formed, which completely fills the wound site, forming a mechanical barrier against bacterial infection. The powder does not exhibit the characteristic fly-away when being applied to the wound site, and administration is perfected due to the precise powder placement.

The gel form of the composition is especially useful in wounds with lesser amounts of exudate, in burns, and in surgical sites. Application of the gel can be dispensed through a tube, a syringe, or the reservoir in a topical patch. The gel can be made of about 1-75 wt. % hydrolyzed collagen and about 1-99 wt. % water. It is preferable to use about 60 wt. % collagen. The gel can be formed by adding sterile water to the powder. The gel has the added advantage of adding moisture to the wound site, as well as inherent bacteriostatic properties, and stays positioned where applied.

A film form of the medicament composition may be made by mixing the powdered form with deionized water under heat at 155-175° F. Cross-linking and other agents, such as humectant, propylene glycol, sorbitol, and glycerine, may be added to the mixture. A preservative (such as benzyl alcohol or paraben) can be added. The mixture is cast on a belt liner by knife on a roll coating machine to form a liquid film, which is oven-dried. The film form can also be formed by cooling the liquid solution. These films can be used for drug or other chemical delivery, especially in dental applications. Antimicrobial and other medicinal agents can also be added to the film as needed for specific applications.

The composition may be formulated as a nutritional supplement. For example, at least one of vitamin A, vitamin C, vitamin D, vitamin E, vitamin B12, magnesium oxide, chelated manganese, grape seed extract, zinc, an alginate, cellulose, honey, chromium picolinate, selenium, glutamine, and glycosaminoglycans can be added to the composition to produce a nutrient composition for oral intake.

The composition can be used as an excellent drug vehicle system including acidic, neutral or complexed drug medications.

### Methods of Wound Healing and Tissue Repair

The method of treating a wound may include administering at least one of the compositions disclosed above to a subject in need thereof.

In an embodiment, subjects having heavily exudative or wet wounds can be treated, at least initially, with a highly hydrophilic composition, including at least about 50% by weight bovine sourced hydrolyzed collagen (e.g., about 50% to about 60% by weight bovine sourced hydrolyzed collagen), at least about 20% by weight marine sourced hydrolyzed collagen (e.g., about 20% to about 30% by weight marine sourced hydrolyzed collagen), and up to about 30% by weight hydrolyzed whey protein (e.g., about 5% to about 30% by weight hydrolyzed whey protein). Elastin may be added to this composition (e.g., up to about 20%) during the closing phase of wound treatment to improve tensile strength and reduce scar formation. Some or all of the hydrolyzed collagen in this composition may be LMW hydrolyzed collagen.

In a further embodiment, subjects having dry wounds can be treated with a composition including at least about 50% by weight marine sourced hydrolyzed collagen, about 20% by weight or less hydrolyzed whey, about 10% by weight or less bovine sourced hydrolyzed collagen, and about 5% by weight or less elastin. Some or all of the hydrolyzed collagen in this composition may be LMW hydrolyzed collagen.

In a further embodiment, subjects may be administered a composition including bovine sourced hydrolyzed collagen, marine sourced hydrolyzed collagen and hydrolyzed whey. The composition may further include about 70% bovine sourced hydrolyzed collagen, about 20% marine sourced hydrolyzed collagen, and about 10% bovine derived hydrolyzed whey. Some or all of the hydrolyzed collagen in this composition may be LMW hydrolyzed collagen.

In a further embodiment, a composition including bovine sourced hydrolyzed collagen, marine sourced hydrolyzed collagen and hydrolyzed whey may be administered as a medical food to a subject in need thereof. The composition may include about 70% bovine sourced hydrolyzed collagen, about 20% marine sourced hydrolyzed collagen, and about 10% bovine derived hydrolyzed whey. Some or all of the hydrolyzed collagen in this composition may be LMW hydrolyzed collagen.

In a further embodiment, a composition including collagen and bioactive glass may be administered to a subject in need thereof. The composition may include between 0.1% to 50% bioactive glass. Some or all of the collagen in this composition may be LMW collagen. Some or all of the collagen in this composition may be hydrolyzed collagen.

It is to be understood that the wound healing and tissue repair composition and method of using low molecular weight hydrolyzed collagen is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A composition for wound healing or tissue repair comprising low molecular weight collagen and bioactive glass, wherein the low molecular weight collagen has a molecular weight of about 10-1,000 Daltons.

2. The composition of claim 1, wherein the composition comprises about 0.1%-2.0% by weight (?) bioactive glass.

3. The composition of claim 1, wherein the composition comprises about 2.0% by weight bioactive glass.

4. The composition of claim 1, wherein the composition comprises about 0.1%-10.5% by weight bioactive glass.

5. The composition of claim 1, wherein the composition comprises about 1.0%-50% by weight bioactive glass.

6. The composition of claim 1, wherein the bioactive glass has an average particle size between about 10 μm and about 90 μm.

7. The composition of claim 1, wherein the bioactive glass comprises about 45 wt % SiO₂, about 24.5 wt % CaO, about 24.5 wt % Na₂O, and about 6.0 wt % P₂O₅.

8. The composition of claim 1, wherein the low molecular weight collagen has a molecular weight of about 500 Da-100 Da.

9. The composition of claim 1, wherein the low molecular weight collagen has a molecular weight of about 400 Da-100 Da.

10. A composition for wound healing or tissue repair comprising bioactive glass and hydrolyzed collagen, the hydrolyzed collagen comprising bovine sourced hydrolyzed collagen, marine sourced hydrolyzed collagen, and hydrolyzed whey, wherein one or more of the bovine sourced hydrolyzed collagen and the marine sourced hydrolyzed collagen comprise low molecular weight hydrolyzed collagen having a molecular weight of about 100-1,000 Daltons.

11. The composition of claim 10, wherein the hydrolyzed collagen comprises about 50%-70% by weight bovine sourced hydrolyzed collagen, about 20% by weight marine sourced hydrolyzed collagen, and about 10% by weight hydrolyzed whey.

12. The composition of claim 10, wherein the composition comprises about 0.1%-2.0% by weight bioactive glass.

13. The composition of claim 10, wherein the composition comprises by weight 2.0% bioactive glass.

14. The composition of claim 10, wherein the composition comprises about 0.1%-10.5% by weight bioactive glass.

15. The composition of claim 10, wherein the composition comprises about 1.0%-20% by weight bioactive glass.

16. The composition of claim 10, wherein the bioactive glass has an average particle size between about 10 μm and about 90 μm.

17. The composition of claim 10, wherein the bioactive glass comprises about 45 wt % SiO₂, about 24.5 wt % CaO, about 24.5 wt % Na₂O, and about 6.0 wt % P₂O₅.

18. The composition of claim 10, wherein the low molecular weight collagen has a molecular weight of about 500 Da-100 Da.

19. The composition of claim 10, wherein the low molecular weight collagen has a molecular weight of about 400 Da-100 Da.

20. A method of treating a wound comprising administering a composition to a subject in need thereof, wherein the composition comprises bioactive glass and low molecular weight collagen, wherein the low molecular weight collagen has a molecular weight of about 100-1,000 Daltons.
